(19) United States
(12) Patent Application Publication
(10) Pub. No.: US 2015/0051492 A9
(43) Pub. Date: Feb. 19, 2015

(54) ULTRASONIC PROBE AND ULTRASONIC DIAGNOSTIC APPARATUS

(71) Applicant: GE Medical Systems Global Technology Company, LLC, Waukesha, WI (US)

(72) Inventors: Shinichi Amemiya, Tokyo (JP); Masaki Tsuda, Tokyo (JP)

(73) Assignee: GE MEDICAL SYSTEMS GLOBAL TECHNOLOGY COMPANY, LLC, Waukesha, WI (US)

(21) Appl. No.: 14/078,150

(22) Filed: Nov. 12, 2013

(30) Foreign Application Priority Data
Nov. 14, 2012 (JP) 2012-250056

(51) Int. Cl. A61B 8/00

(52) U.S. Cl. CPC A61B 8/4444
USPC 600/459; 29/594

(57) ABSTRACT

An ultrasonic probe configured to be connected to an apparatus main body of an ultrasonic diagnostic apparatus that is configured to perform an ultrasonic procedure on a subject is provided. The ultrasonic probe includes a transducer configured to transmit an ultrasonic wave to the subject and receive an ultrasonic echo reflected from the subject, an inductor connected in parallel with the transducer, and a current limiter connected to the inductor and configured to limit a current flowing through the inductor to a predetermined current level or lower.

### CROSS REFERENCE TO RELATED APPLICATIONS

This application claims the benefit of Japanese Patent Application No. 2012-250056 filed Nov. 14, 2012, which is hereby incorporated by reference in its entirety.

### BACKGROUND OF THE INVENTION

The present invention relates to an ultrasonic probe and an ultrasonic diagnostic apparatus equipped with the ultrasonic probe.

An ultrasonic diagnostic apparatus transmits an ultrasonic wave from a plurality of transducers provided in an ultrasonic probe to a subject and receives an ultrasonic echo reflected from the subject at each transducer. Each transducer is connected to an inductor for adjusting a frequency of the ultrasonic wave.

Japanese Unexamined Patent Application Publication No. 2003-339700 discloses a switching circuit in which each of transducers Tr for a low frequency, a medium frequency, and a high frequency is connected to a single inductor, which switching circuit selectively drives them. Japanese Unexamined Patent Application Publication No. 2003-339700 also discloses a switching circuit in which two inductors for the low frequency and the high frequency are connected to a single transducer in series, which switching circuit selectively drives them.

However, with more inductors prepared in a transducer, a volume (or area) occupied by the inductor increases. Thus, to apply a high current especially during transmission, a rated current of the inductor must be high, and therefore the inductor must be arranged in a connector or the like outside the ultrasonic probe instead of being built in the ultrasonic probe, as shown in FIG. 4 of Japanese Unexamined Patent Application Publication No. 2003-339700. Furthermore, in order for an operator to easily handle the ultrasonic probe, downsizing of the ultrasonic probe has been desired recently. It is becoming harder to build an inductor in such an ultrasonic probe that is becoming smaller.

### BRIEF DESCRIPTION OF THE INVENTION

The disclosure provides a downsized ultrasonic probe and an ultrasonic diagnostic apparatus with the ultrasonic probe using a current limiter and an inductor.

In a first aspect, an ultrasonic probe connected to an apparatus main body of an ultrasonic diagnostic apparatus that performs an ultrasonic diagnose on a subject is provided. The ultrasonic probe includes a transducer Tr that transmits an ultrasonic wave to a subject and receives an ultrasonic echo reflected from the subject, an inductor connected to the transducer Tr in parallel, and a current limiter that is connected to the inductor and limits a current flowing through the inductor to a predetermined current level or lower.

The inductor adjusts the frequency of the ultrasonic echo. The current limiter also prevents the inductor from operating when transmitting the ultrasonic wave, and the current limiter causes the inductor to operate when transmitting the ultrasonic wave.

The ultrasonic probe further includes a cable for transferring the ultrasonic echo received by the ultrasonic probe to the apparatus main body, and the inductor and the current limiter are arranged on an ultrasonic probe side of the cable or on the apparatus main body side of the cable.

Furthermore, the transducer is arranged in an array direction in one dimension or arranged in both the array direction and an elevation direction perpendicular to the array direction in two dimensions.

In a second aspect, an ultrasonic diagnostic apparatus including the ultrasonic probe of the first aspect and an ultrasonic diagnostic apparatus main body connected to the ultrasonic probe is provided.

The systems and methods described herein can provide a downsized ultrasonic probe using the current limiter and the inductor.

### BRIEF DESCRIPTION OF THE DRAWINGS

FIG. 1 is a schematic diagram showing an example of an embodiment of ultrasonic image diagnostic apparatus.

FIG. 2 is a block diagram showing an ultrasonic probe 20.

FIGS. 3A and 3B are arrangement examples of a frequency adjustment circuit 10.

FIGS. 4A and 4B are examples of a current limiting circuit.

### DETAILED DESCRIPTION OF THE INVENTION

### First Embodiment

(Configuration of Ultrasonic Diagnostic Apparatus)

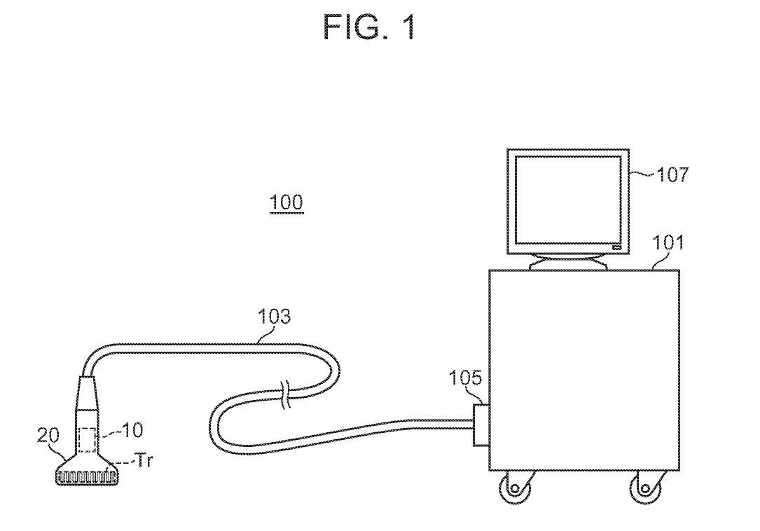

FIG. 1 is a schematic diagram showing an example of an ultrasonic diagnostic apparatus 100 according to a first embodiment. As shown in FIG. 1, the ultrasonic diagnostic apparatus 100 has an ultrasonic diagnostic apparatus main body 101 and an ultrasonic probe 20 connected to the ultrasonic diagnostic apparatus main body. The ultrasonic probe 20 is connected to the ultrasonic diagnostic apparatus main body 101 via a cable 103 and a connector 105.

The ultrasonic probe 20 is provided with a transducer Tr and a frequency adjustment circuit 10. The transducer Tr transmits an ultrasonic wave upon receiving an ultrasonic wave transmission signal from a transmission unit (not shown). The transducer Tr also receives an ultrasonic echo reflected from the subject. The ultrasonic echo is transmitted to a receiving unit (not shown) via the frequency adjustment circuit 10.

An ultrasonic echo signal is input from the frequency adjustment circuit 10 to the ultrasonic diagnostic apparatus main body 101 via the cable 103 and the connector 105. The ultrasonic echo signal is A/D converted by an A/D conversion unit in the apparatus main body 101, which is not shown. The ultrasonic diagnostic apparatus 100 then generates an ultrasonic image based on the A/D converted ultrasonic echo signal, and the ultrasonic image is displayed on a display unit 107 of the ultrasonic diagnostic apparatus main body 101.

(Configuration of Ultrasonic Probe)

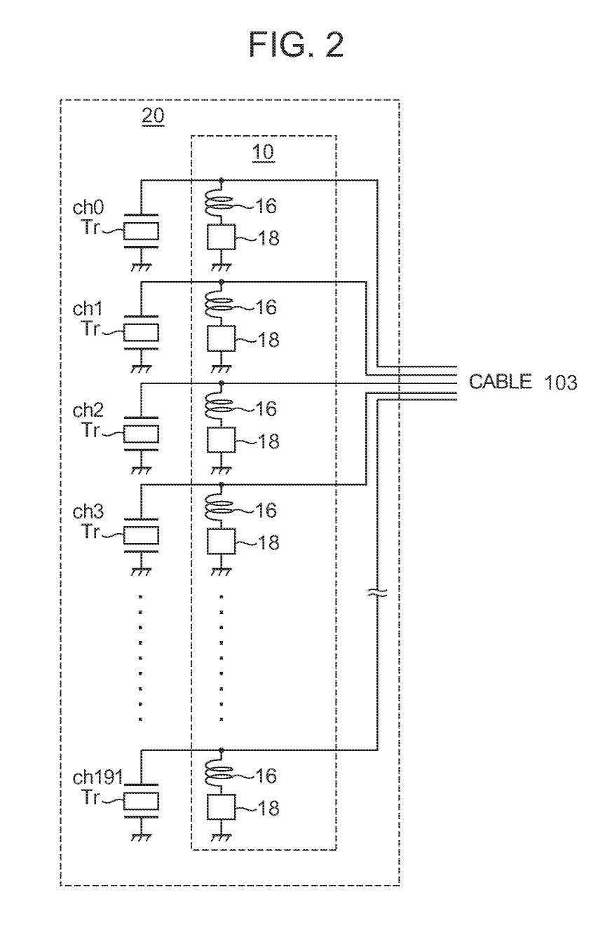

FIG. 2 is a block diagram showing the ultrasonic probe 20. The ultrasonic probe 20 has a plurality of transducers Tr provided to a channel 0 (ch0) to a channel 191 (ch191). Each transducer Tr has an inductor 16 arranged in parallel. The inductor 16 is connected in series to a current limiting circuit 18 that limits the current flowing through the inductor 16. The inductor 16 has a role of adjusting a resonance frequency of the transducer Tr and improving a signal-to-noise ratio of the ultrasonic echo signal received by the transducer Tr.

As the inductor 16, a small inductor 16 is used to decrease the size of the ultrasonic probe 20. For example, an SMD (Surface Mounted Device) type of the inductor 16 may have a size of approximately 1.8 mm (L) * 0.8 mm (W) * 0.8 mm (H). Such a small inductor 16 has a rated current of, for example, 0.01 Amps (A).

On the other hand, as the higher current is applied to the transducer Tr, the more ultrasonic wave can be transmitted to the subject. Thus, when transmitting the ultrasonic wave to the subject, the inductor 16 arranged in parallel with the transducer Tr is applied with a current of, for example, 0.6 A. Therefore, the inductor 16 with the rated current of, for example 0.7 A, must be prepared. The size of the inductor 16 with the rated current of 0.7 A may be approximately 4.0 mm (L) * 4.0 mm (W) * 1.8 mm (H). If the inductor 16 of this size is arranged in all of the 192 channels, the ultrasonic probe 20 would be too large.

In this embodiment, the current limiting circuit 18 is connected to the small inductor 16 in series. As described above, the volume of the inductor 16 with the rated current of 0.01 A is approximately 1/25 of the volume of the inductor 16 with the rated current of 0.7 A. In comparison including both the volume of the inductor 16 with the rated current of 0.01 A and the volume of the current limiting circuit 18, it is still about ¼ to ⅓. Thus, the configuration of this embodiment facilitates decreasing the size of the ultrasonic probe 20.

According to the embodiment, the current limiting circuit 18 is set with the upper limit of, for example, 0.01 A and, when a current higher than 0.01 A flows into the inductor 16, the current from the inductor 16 to a ground is cut off. Accordingly, when transmitting the ultrasonic wave to the subject, the transmission unit (not shown) lets the current of, for example, 0.6 A flow through the transducer Tr, while the current limiting circuit 18 blocks the current so as not to operate the inductor 16. Thus, the inductor 16 is protected against an overcurrent. On the other hand, when receiving the ultrasonic echo reflected from the subject, the ultrasonic echo signal from the transducer Tr has a current of 0.01 A or lower and the current limiting circuit 18 causes the inductor 16 to operate. The inductor 16 thus improves the signal-to-noise ratio of the ultrasonic echo signal.

This embodiment shows an example in which the 192 transducers Tr are arrayed in the array direction. It is a so-called 1-D array oscillator. However, the embodiment is not limited to the 1-D array oscillator. Although not illustrated, the frequency adjustment circuit 10 (the inductor 16 and the current limiting circuit 18) may be arranged in parallel with 1.25-D array oscillator. The 1.25-D array oscillator is constituted by a plurality of transducers Tr arrayed in pairs in the elevation direction (the direction perpendicular to the array direction), in which a signal is supplied to a pair of transducers Tr in the elevation direction by a pair selection by an unshown switch circuit. Although not illustrated, this embodiment may be applied to a 1.5-D array oscillator. The 1.5-D array oscillator is constituted by transducers Tr arrayed in the elevation direction, in which the transducer Tr is controlled not in pair but independently in the elevation direction.

(Configuration of Frequency Adjustment Circuit)

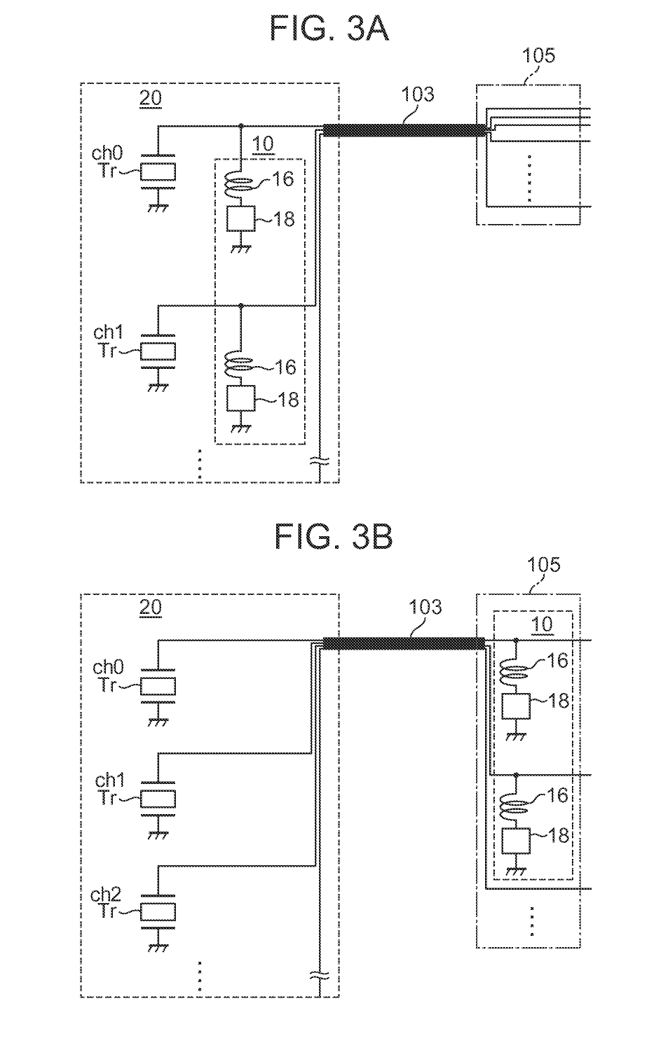

FIGS. 3A and 3B are block diagrams showing arrangement positions of the frequency adjustment circuit 10. FIG. 3A is an arrangement example of the frequency adjustment circuit 10 explained with reference to FIG. 2. FIG. 3B is another arrangement example of the frequency adjustment circuit 10.

In FIG. 3A, the frequency adjustment circuit 10 is built in the ultrasonic probe 20. The ultrasonic echo signal is connected to the apparatus main body 101 via the cable 103 and the connector 105. The connector 105 includes wiring alone and the frequency adjustment circuit 10 is not built therein.

On the other hand, in FIG. 3B, the frequency adjustment circuit 10 is built in the connector 105. The ultrasonic echo signal received by the transducer Tr is transmitted to the connector 105 via the cable 103. The ultrasonic echo then improves the signal-to-noise ratio in the frequency adjustment circuit 10 in the connector 105. Because the ultrasonic probe 20 does not have the inductor 16 or the current limiting circuit 18, the size of the ultrasonic probe 20 is further decreased.

(Configuration of Current Limiting Circuit)

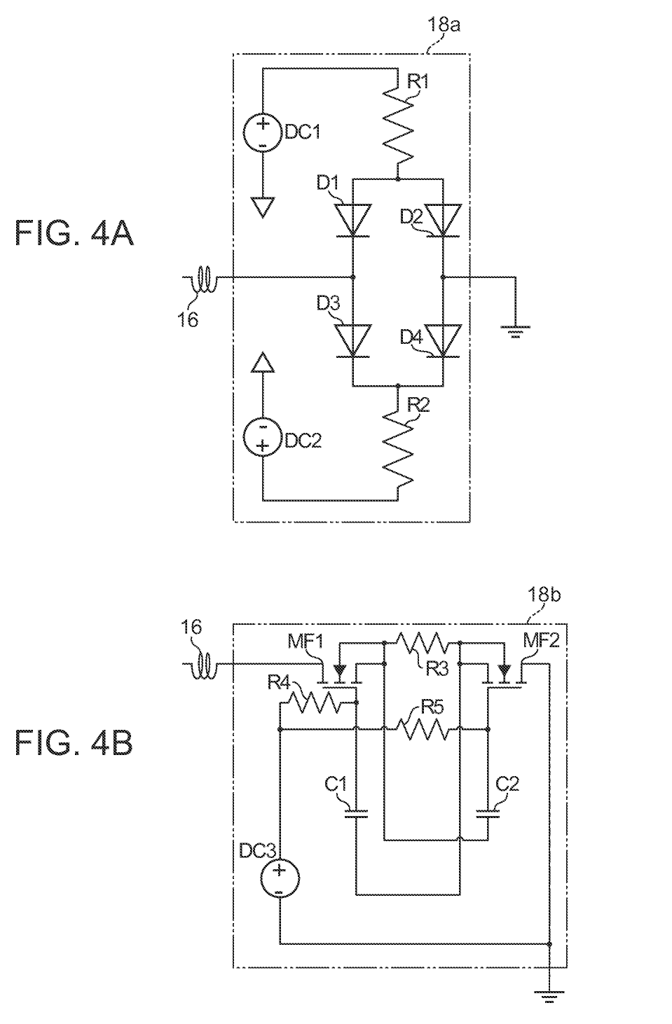

FIGS. 4A and 4B are block diagrams showing the current limiting circuit 18. FIG. 4A is an example of a current limiting circuit 18a using a diode. FIG. 4B is an example of a current limiting circuit 18b using a MOSFET (Metal-Oxide-Semiconductor Field-Effect Transistor). Both the current limiting circuit 18a and the current limiting circuit 18b have a function of blocking a current when an overcurrent flows through the inductor 16.

The current limiting circuit 18a shown in FIG. 4A is connected to the inductor 16 at one end thereof and to the ground at the other end. DC power sources DC1 and DC2 are provided, and a resistor R1 connected in series to a +side of the DC power source DC1 is connected to anodes of the PN connection diodes D1 and D2. The PN connection diodes D1 and D2 are arranged in parallel, with a cathode of the PN connection diode D1 being connected to the inductor 16 and a cathode of the PN connection diode D2 being connected to the ground. Moreover, a resistor R2 connected in series to the +side of the DC power source DC2 is connected to cathodes of PN connection diodes D3 and D4. The PN connection diodes D3 and D4 are arranged in parallel, with an anode of the PN connection diode D3 being connected to the inductor 16 and an anode of the PN connection diode D4 being connected to the ground.

By the settings of the resistors R1 and R2 and the DC power sources DC1 and DC2, the flow of the current is blocked when the current higher than the predetermined level flows through the inductor 16.

The current limiting circuit 18b shown in FIG. 4B has two MOSFETs (MF1 and MF2), with a drain of the MOSFET (MF1) being connected to the inductor 16 and a source of the MOSFET (MF2) being connected to the ground. A source of the MOSFET (MF1) and a drain of the MOSFET (MF2) are connected by a resistor R3. A capacitor C1 connected to a gate of the MOSFET (MF1) is arranged in parallel with the resistor R3, and a capacitor C2 connected to a gate of the MOSFET (MF2) is also arranged. The gate of the MOSFET (MF1) is connected to a DC power source DC3 via a resistor R4, and the gate of the MOSFET (MF2) is connected to the DC power source DC3 via a resistor R5.

1. An ultrasonic probe configured to be connected to an apparatus main body of an ultrasonic diagnostic apparatus that is configured to perform an ultrasonic procedure on a subject, the ultrasonic probe comprising:
a transducer configured to transmit an ultrasonic wave to the subject and receive an ultrasonic echo reflected from the subject;
an inductor connected in parallel with the transducer; and
a current limiter connected to the inductor and configured to limit a current flowing through the inductor to a predetermined current level or lower.

2. The ultrasonic probe according to claim 1, wherein the inductor is configured to adjust the ultrasonic echo.

3. The ultrasonic probe according to claim 1, wherein the current limiter is configured to prevent the inductor from operating when transmitting the ultrasonic wave and the current limiter is configured to cause the inductor to operate when receiving the ultrasonic wave.

4. The ultrasonic probe according to claim 2, wherein the current limiter is configured to prevent the inductor from operating when transmitting the ultrasonic wave and the current limiter is configured to cause the inductor to operate when receiving the ultrasonic wave.

5. The ultrasonic probe according to claim 1,
wherein the ultrasonic probe includes a cable configured to transfer the ultrasonic echo received by the ultrasonic probe to the apparatus main body, and
the inductor and the current limiter are arranged on an ultrasonic probe side of the cable or on an apparatus main body side of the cable.

6. The ultrasonic probe according to claim 2,
wherein the ultrasonic probe includes a cable configured to transfer the ultrasonic echo received by the ultrasonic probe to the apparatus main body, and
the inductor and the current limiter are arranged on an ultrasonic probe side of the cable or on an apparatus main body side of the cable.

7. The ultrasonic probe according to claim 3,
wherein the ultrasonic probe includes a cable configured to transfer the ultrasonic echo received by the ultrasonic probe to the apparatus main body, and
the inductor and the current limiter are arranged on an ultrasonic probe side of the cable or on an apparatus main body side of the cable.

8. The ultrasonic probe according to claim 4,
wherein the ultrasonic probe includes a cable configured to transfer the ultrasonic echo received by the ultrasonic probe to the apparatus main body, and
the inductor and the current limiter are arranged on an ultrasonic probe side of the cable or on an apparatus main body side of the cable.

9. The ultrasonic probe according to claim 1, wherein the transducer is arranged in an array direction or arranged in both the array direction and an elevation direction perpendicular to the array direction.

10. An ultrasonic diagnostic apparatus including:
an ultrasonic probe according to claim 1; and
an ultrasonic diagnostic apparatus main body connected to the ultrasonic probe.

11. An ultrasonic diagnostic apparatus including:
an ultrasonic probe according to claim 2; and
an ultrasonic diagnostic apparatus main body connected to the ultrasonic probe.

12. An ultrasonic diagnostic apparatus including:
an ultrasonic probe according to claim 3; and
an ultrasonic diagnostic apparatus main body connected to the ultrasonic probe.

13. An ultrasonic diagnostic apparatus including:
an ultrasonic probe according to claim 4; and
an ultrasonic diagnostic apparatus main body connected to the ultrasonic probe.

14. An ultrasonic diagnostic apparatus including:
an ultrasonic probe according to claim 5; and
an ultrasonic diagnostic apparatus main body connected to the ultrasonic probe.

15. An ultrasonic diagnostic apparatus including:
an ultrasonic probe according to claim 6; and
an ultrasonic diagnostic apparatus main body connected to the ultrasonic probe.

16. An ultrasonic diagnostic apparatus including:
an ultrasonic probe according to claim 7; and
an ultrasonic diagnostic apparatus main body connected to the ultrasonic probe.

17. An ultrasonic diagnostic apparatus including:
an ultrasonic probe according to claim 8; and
an ultrasonic diagnostic apparatus main body connected to the ultrasonic probe.

18. An ultrasonic diagnostic apparatus including:
an ultrasonic probe according to claim 9; and
an ultrasonic diagnostic apparatus main body connected to the ultrasonic probe.

19. A method of assembling an ultrasonic probe, the method comprising:
coupling a transducer in parallel with an inductor, wherein the transducer is configured to transmit an ultrasonic wave to a subject and receive an ultrasonic echo reflected from the subject;
coupling a current limiter to the inductor, wherein the inductor is configured to limit a current flowing through the inductor to a predetermined current level or lower.

20. The method according to claim 19, further comprising arranging the inductor and the current limiter on a cable configured to transfer the ultrasonic echo received by the ultrasonic probe to an apparatus main body.
